(19) United States
(12) Patent Application Publication
(10) Pub. No.: US 2007/0106066 A1
(43) Pub. Date: May 10, 2007

(54) Cherkasky fusion proteins containing antibody-, antigen- and microtubule-binding regions and immune response-triggering regions

(76) Inventor: Alexander Cherkasky, Dusseldorf (DE)

Correspondence Address: BROOKS KUSHMAN P.C., 1000 TOWN CENTER, TWENTY-SECOND FLOOR, SOUTHFIELD, MI, 48075, US

(21) Appl. No.: 10/577,613

(22) PCT Filed: Oct. 28, 2004
(86) PCT No.: PCT/IB04/03536

§ 371 Date: Apr. 28, 2006

(30) Foreign Application Priority Data
Oct. 28, 2003 (DE) 10350131.2
Oct. 28, 2003 (DE) 10350122.3

(51) Int. Cl. C07K 14/54; C07K 14/52; C07H 21/04

(52) U.S. Cl. 530/351; 536/23.5

(57) ABSTRACT

The invention relates to the fields of tumour physiology and biotechnology. The object of the invention is to develop effective and selective novel fusion proteins and fusion protein-antibody complexes against various types of leukaemia and solid tumours. Selectivity is achieved by cell-specific or tumour-specific ligands of the fusion proteins or by antibodies of the fusion protein-antibody complexes. Effectiveness is achieved on the one hand by the direct binding of the microtubules or cytoskeleton elements to the microtubule-binding regions and on the other hand by induction, as well as by the reinforcement of the immune reaction by regions that trigger the immune reaction on the target cells.

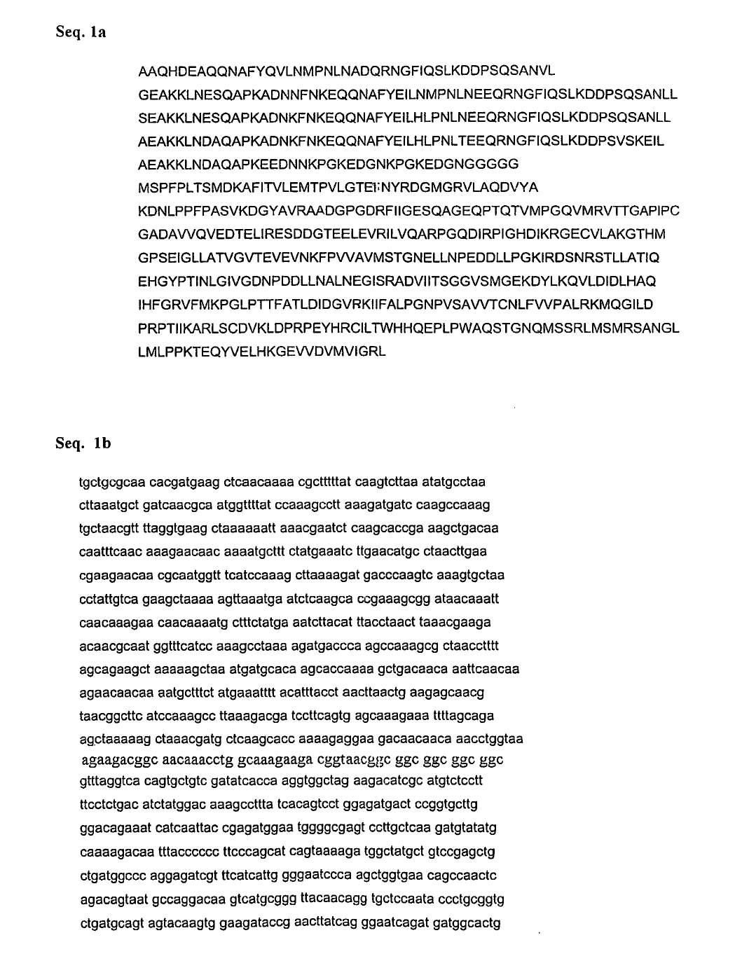

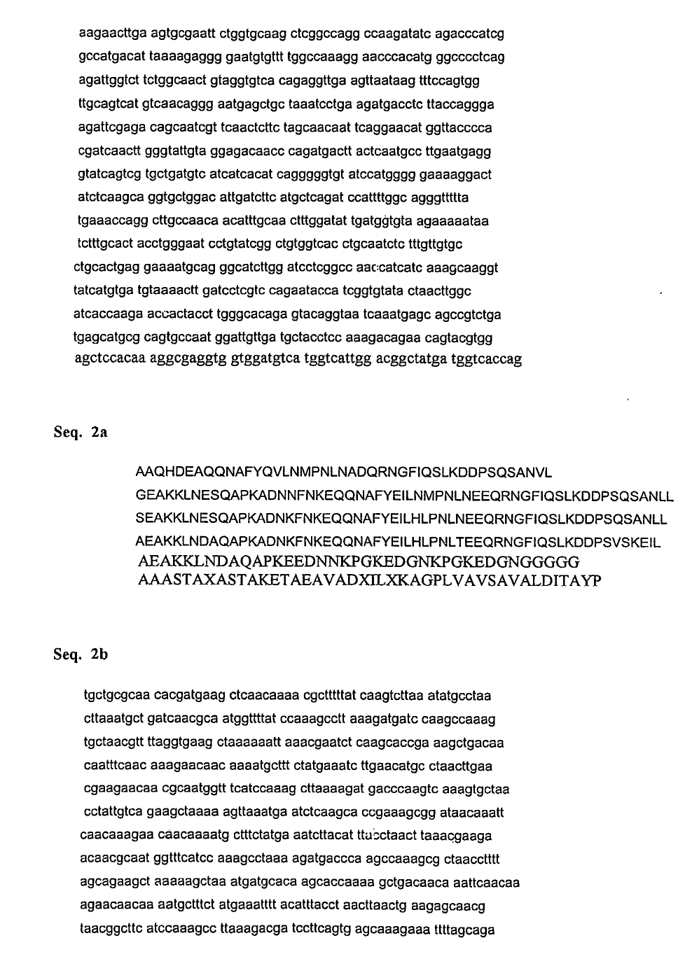

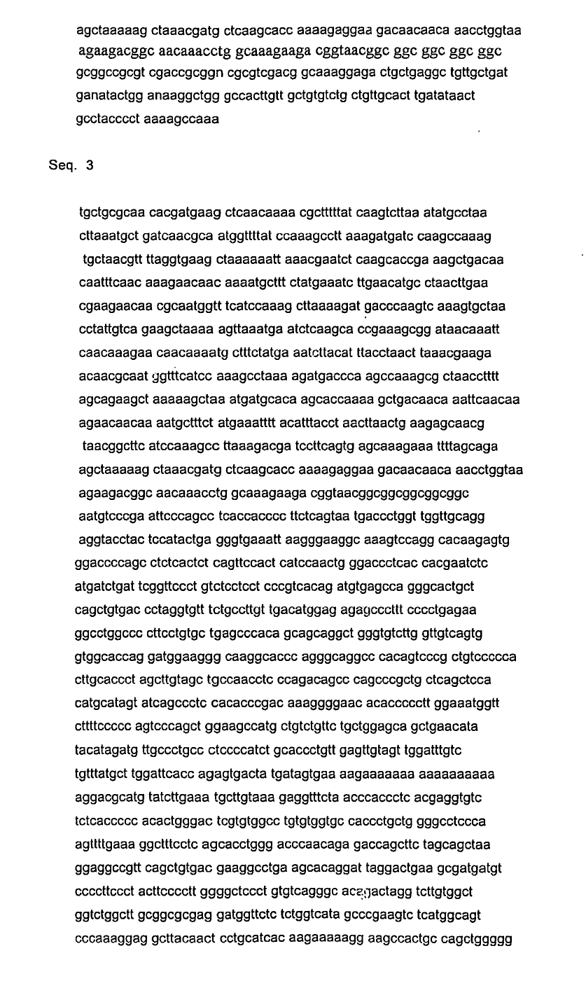

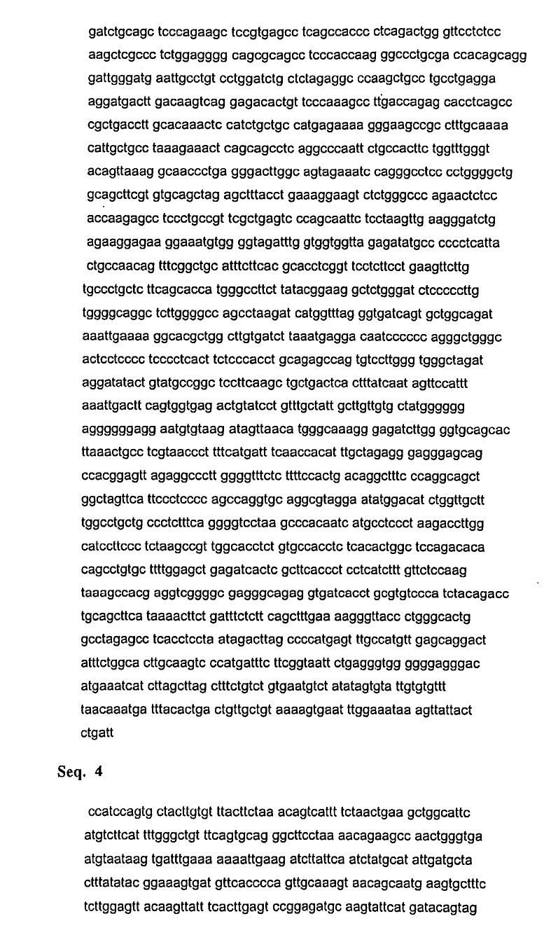

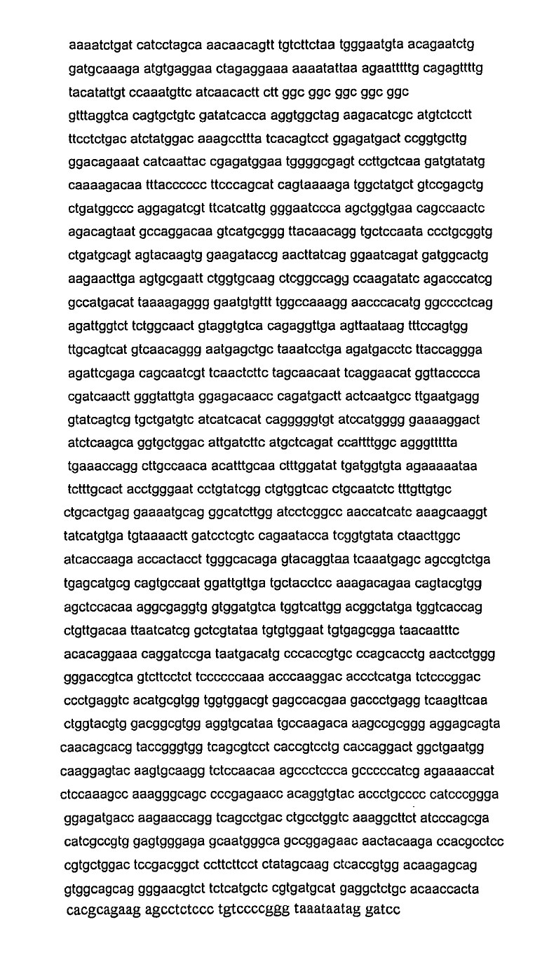

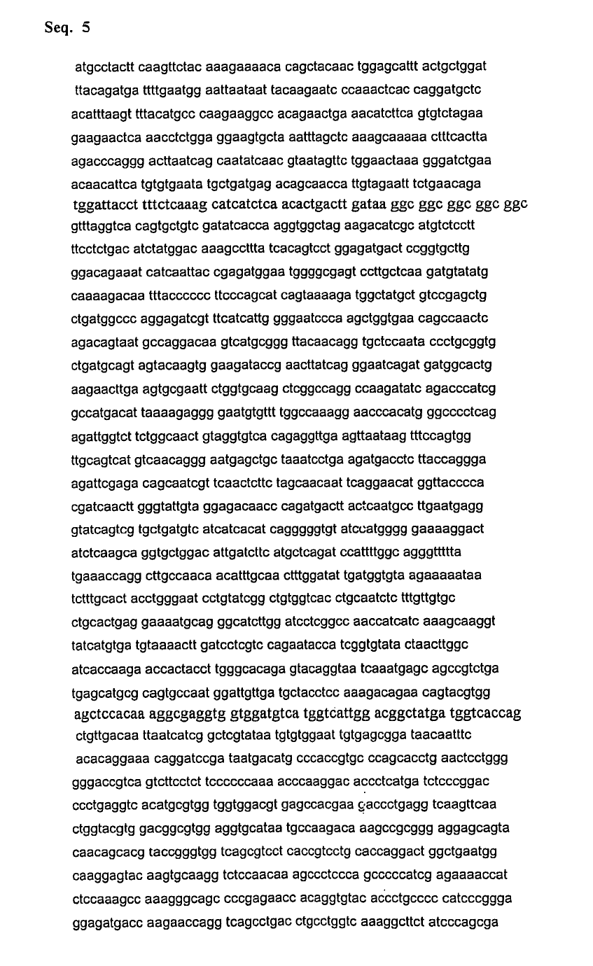

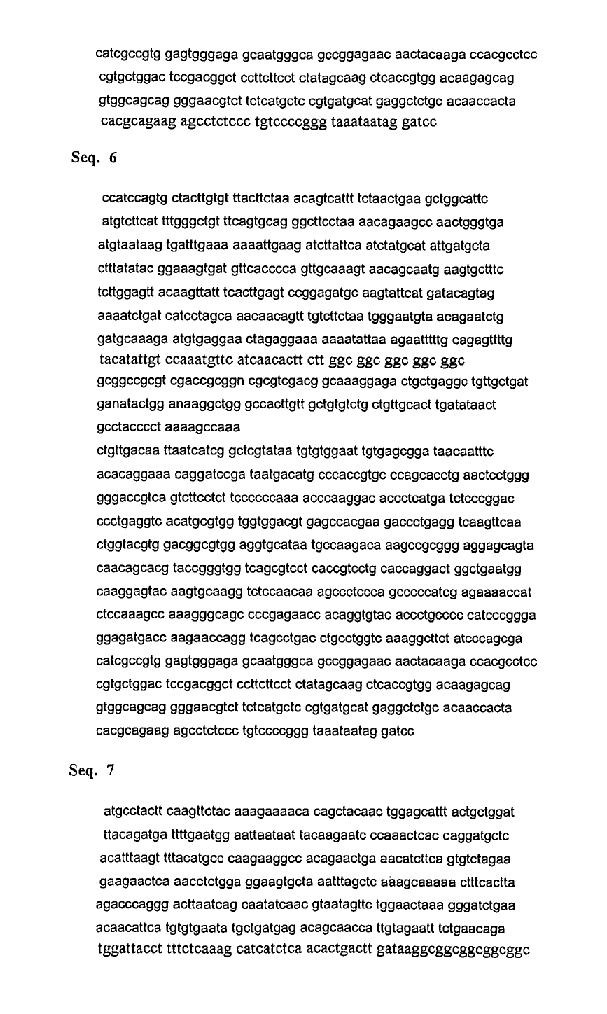

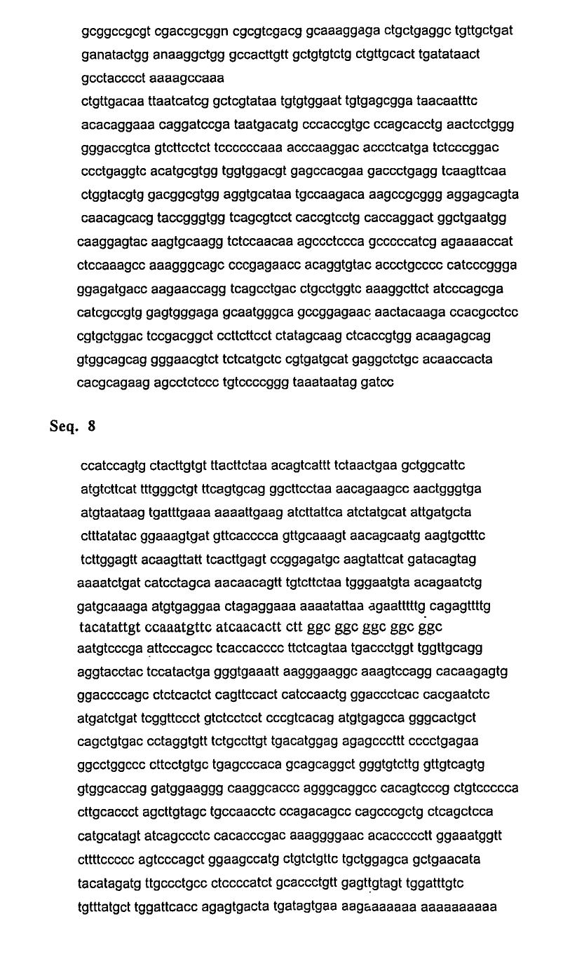

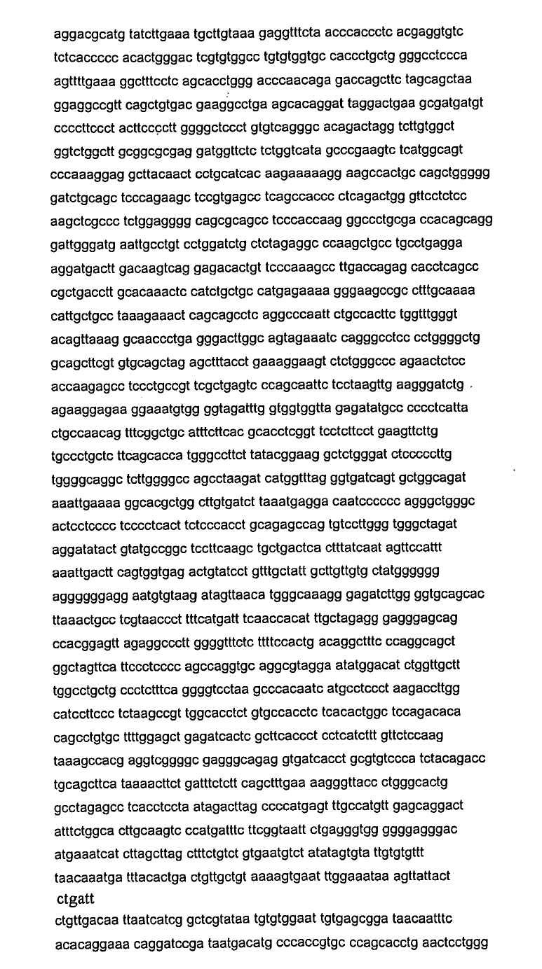

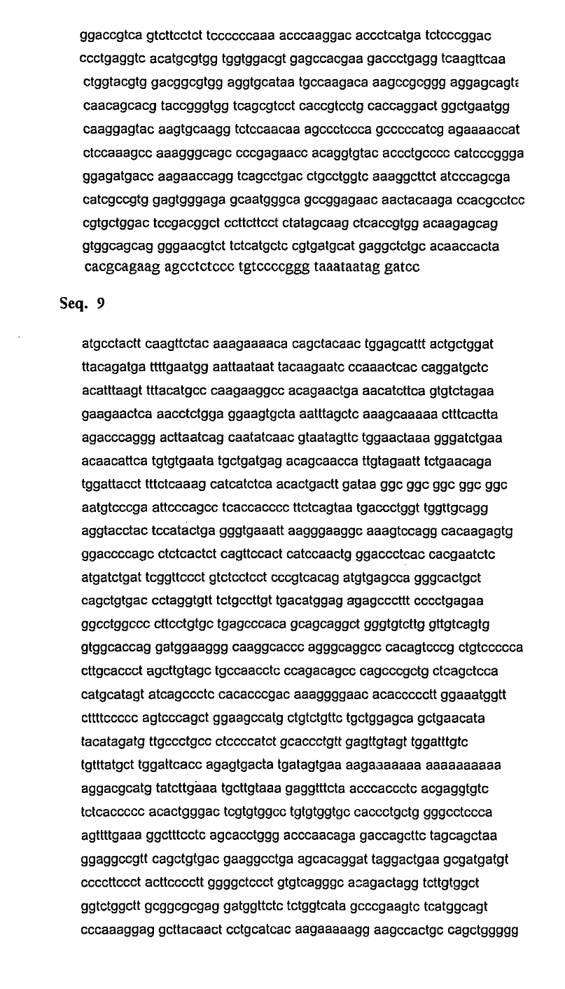

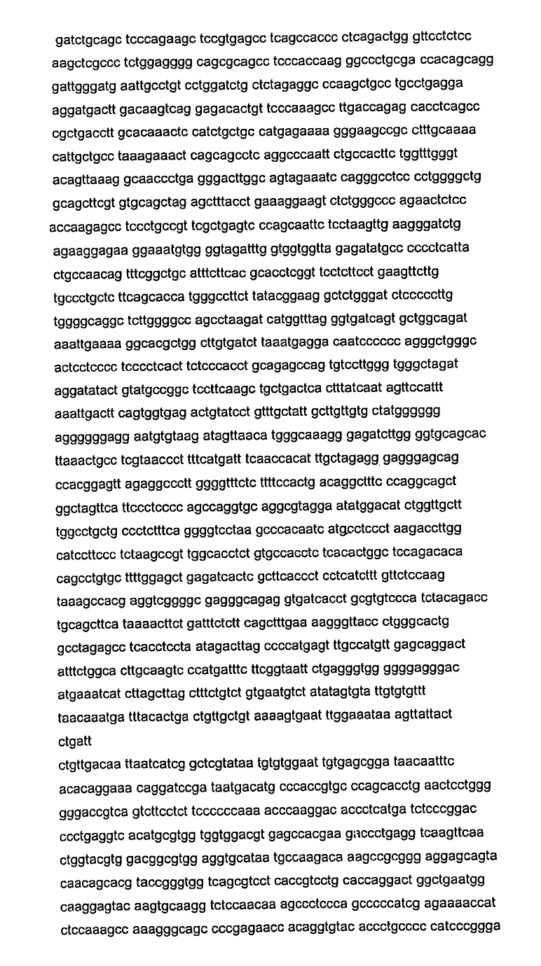

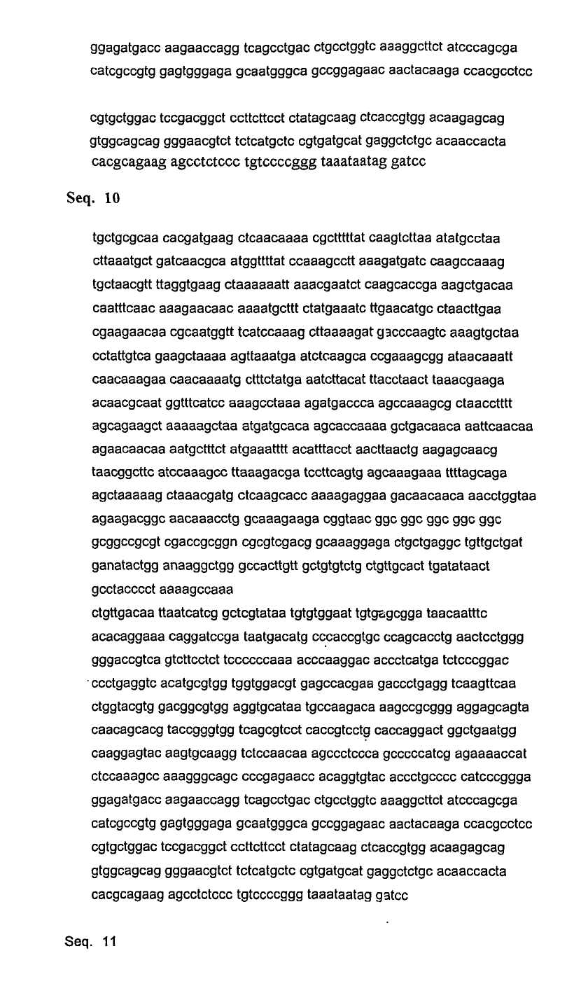

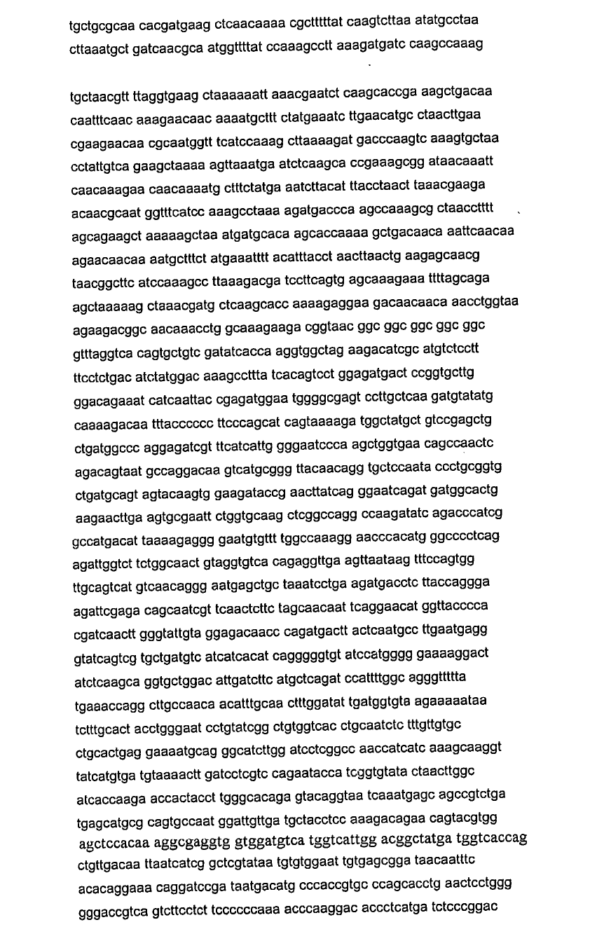

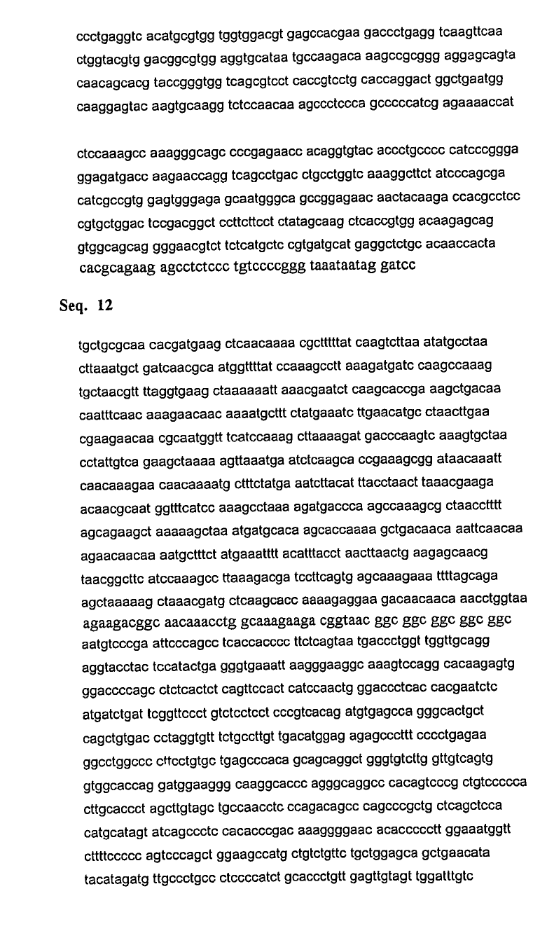

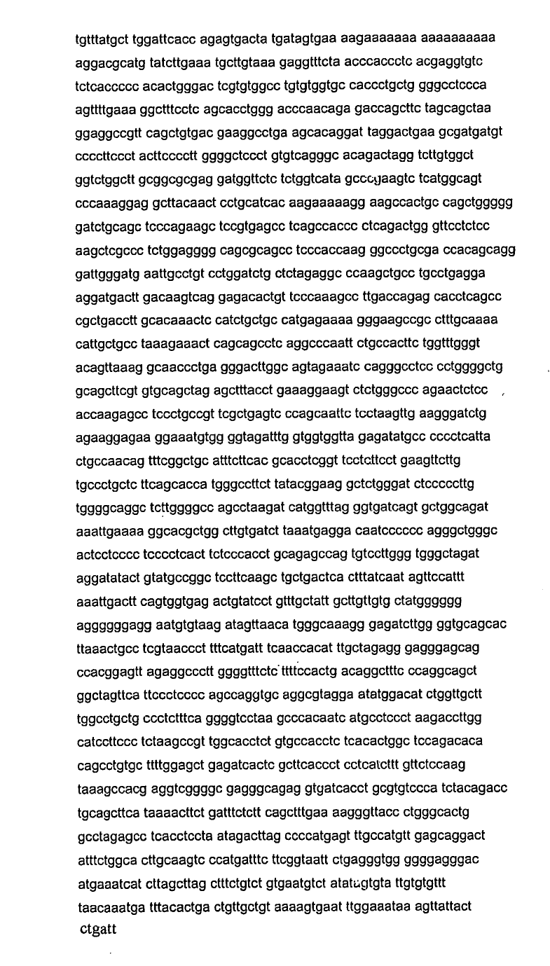

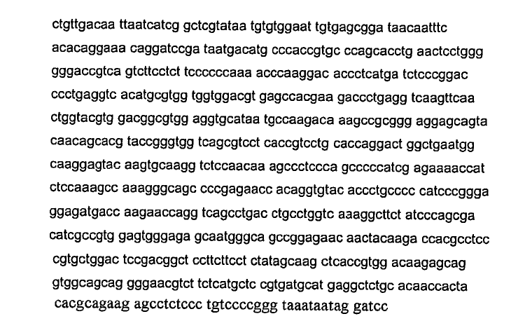

### BACKGROUND OF THE INVENTION

The invention relates to the fields of tumour physiology and biotechnology.

Operations, irradiation and chemotherapy are current important measures in the tumor therapy. By the chemotherapy will be used cytostatic molecules with different mechanisms of action, such as alkylants, nitrosourea-compounds, antagonists of folic acid, pyrimidin and purin-analoga such as fluoruracile, antibiotics with action on DNA-dependent RNA-polymerase or enzymes such as L-Asparaginase. Another group of cytostatic compounds are inhibitions of mitosis such as Taxol and Vinca-alcaloids.

Because of their good antitumor activity, inhibitors of mitosis will be used often. They impact microtubules and so they impact cell division. Colchicin or Vinca-alkaloids bind themselves on specific binding sites of Alpha-and Beta-Tubulines as part of microtubules and inhibit arise of new microtubules. Other inhibitors of mitosis, such as taxol induces destabilization of microtubules.

Inhibitors of mitosis are toxic and therefore problematic for therapeutic use. The toxicity of colchicin is so problematic, that this compound will not be used therapeutically. The alkaloid Taxol, isolated from Taxus is researched intensively.

The most inhibitors of mitosis bind them self on Beta-Tubulin of microtubules. For this binding they possess specific binding sites.

The affinity of these sites is important for classification of inhibitors of mitosis. Between different groups of such compounds such as Colchicin-type, the Taxan type, the Vinca-alkaloid-type or the Rhyoxine-type will be distingnished.

Because of high toxicity of cytostatic compounds, therapies using them trigger many side effects, that can be very difficult and gravy for patients. Therefore, since many years will be searched to find better therapies for avoiding or with reduced side effects. One approach is a strategy to direct acting compounds to target cells.

One of the possibilities to do so, is based on identification of specific epitopes, to produce epitope-specific monoclonal antibodies and than couple the antibodies or antigen-binding fragments of them to therapeutically used molecules.

Such project will be carry out at the University of California, Los Angeles, with the goal of a specific therapy of breast cancer. (Sherie L. Morrison, Ph. D.: “Antibody Fusion Proteins for the Therapy of Breast Cancer”, University of California, Los Angeles, 1997-1999). In this project, antibodies against breast cancer-specific molecules her 2/neu and CEA were fused with immune stimulating molecules, that trigger the activity of T cells.

Altough this approach has an advantage of therapeutic selectivity, it is difficult to realize it in the praxis. Firstly, cell type specific antigens should be found and isolated. They are mostly protein antigens and cell specific epitopes of the antigen will be searched, that have minimally or no similarities to epitopes of proteins of other cell types. This is recessary for avoiding of crossreativity of therapeutically used antibodies. Than, antibodies will be produced against a selected antigen and mostly such procedures as selection and mutagenesis methods such Phage Display are needed, to elect antibodies of high specifity and affinty and with no or reduced cross-reactivity.

Some other complications are modification of non-human antibodies and their humanization. Production of complex antigen-specific elements and their coupling with therapeutically active molecule is expensive and needs necessary expression constructs and suitable expression systems.

So, one of the problems is the more efficient use of antibodies. They are mostly difficult to produce and limited in action, because they activate only macrophages and mostly do not act directly on target cells. One of attempts to improve the action of antibodies are immunotoxins. They are also difficult to produce, to couple, i.e. the coupling of toxins with antibodies is mostly not stable, as well as both internalisation of antibodies is need and they cannot attract additionally the activation of T cells against targets of antibodies coupled with these toxins. The election of toxins is also a problem, because if a toxin will be cleaved from antibody healthy cells can be damaged. One of approaches is to use not toxins, but proteins or their regions available in human cells, but able e.g. as fusion and acting in complexes, to inhibit cell division.

Known are publications in patent literature, in which fusion proteins comprising ligands and microtubule-binding regions, as well as membrane penetration domains, ligands and microtubule-binding domains are described. (DE 199 25 052.9; DE 101 61 899. 9; DE 101 61 738.0 and DE 101 61 739.9).

The disadvantages of them are firstly, that no additionally immune response will be triggered and secondly, choice of tumor-specific antigenes and antigen binding regions is relatively small and a complexation with antibodies is need, with the purpose of their modification, i.e. for making them able, to act directly after penetration of cell membrane and after internalisation and to bind microtubules as compounds of cytoskeleton and its other compounds.

### SUMMARY OF THE INVENTION

The object of the invention is to propose, to develop effective and selective novel fusion proteins and fusion protein-antibody complexes against various types of leukemia and solid tumors. The solution of the problem are the fusion proteins, called Cherkasky fusion proteins containing antibody-, antigen-, and microtubule-binding regions and immune triggering regions. Selectivity is achieved by cell-specific or tumor-specific ligands of the fusion proteins or by antibodies of the fusion protein-antibody complexes.

Effectiveness is achieved on the one hand by the direct binding of the microtubules or cytoskeleton elements to the microtubules-binding regions and on the other hand by induction, as well as by the reinforcement of the immune reaction by regions that trigger the immune reactions on the target cells.

### DESCRIPTION OF THE INVENTION

The fusion proteins, that bind antibodies and build complexes with them contain antibody-binding regions and microtubule-binding regions.

The action of these fusion protein-antibody-complexes is based on the ability of microtubule binding domains to bind microtubules, and grip them, after antibodies recognize and penetrate their target cells.

The antibody binding region is for example Staphylococcal protein A (SPA), excellular region of the Fe receptor CD 64 or other related proteins. The microtubule-binding region is for example gephyrin, putative microtubule binding protein, FLJ 31424 Fis, MID-1, MAP, Tau or other proteins. These fusion proteins can contain long and very long spacer or linker regions such as polyglycine or polyproline. They can preferably be fused to the membrane penetration domaine (MBD) or protein transduction domaine (PTD).

The fusion protein can also contain immune response triggering regions such as Fc, B 7.1 or B 7.2 for increase of action of fusion protein-antibody complexes.

These fusion proteins can contain nucleic acid binding regions, such as for example Rec A and /or polysaccharid binding domains such as, the cellulose binding region of Cip A. The goal thereby is to increase concentration through binding to polymers such biopolymers and so to enhance the action.

These fusion proteins can be fused additionally with GFP or other fluorescent proteins, for optic observation of their action or to measure the concentration according to the intensity of the fluorescence.

In addition, these fusion proteins can contain hinge regions such as for example five glycine regions and at least one GST tag or any other region for carry out of affinity purification.

The meanings of the words “region” and “domaine” are similar.

The group of fusion proteins containing antigen binding; microtubule-binding, and immune response triggering regions also unfold the double action.

The mechanism of action of these fusion proteins is based on their tumor cell specific internalisation and inhibition of cell division via binding of microtubules, as well as through triggering of a tumor cell specific immune response. The microtubule binding region can be elected for example from gephyrin, putative microtubule binding protein, FLJ 31424 Fis, Tau, MID-1 or MAP 1. The immune response triggering region is for example Fc region of an IgG-antibody, B 7.1 or B 7.2 regions for triggering of a T cell response.

The antigen binding regions can be preferably elected from the following proteins: EGF, FGF, CSF, MGF, IL-15, IL-2 and other proteins or their regions.

These fusion proteins can also additionally contain the protein transduction domain (PTD). PTD is in some cases not recessary, in the case if an antigen binding region is a ligand, which will be internalised after interaction with its receptor on the cell surface. In other cases, PTD serves for internalisation of fusion proteins into cells. Examples for PTD or MPD are the gene -3-Protein of the bacteriophage fd, gp 41 or Tat Protein of HIV-1.

The, in the literature described protein transduction domaine (PTD) is a eleven (11) amino acid long region of the HIV Tat protein.

Dowdy and colleagues made fusions of 60 proteins in range of size between 15 kDa and 120 kDa with PTD and than they were denaturated with urea and transported into cells. (Science (285, 1569-1572, 1999) and Nature biotechnology Vol. 17, p. 942, oct. 1999).

After internalisation of the fusion protein, acts the microtubule binding domain such in gephyrin, Tau, MAP or MID-1 in cytosol. It binds microtubules and grips cytoskeleton. The dynamic stability (Wilde et al Nature Cell Biology 2001, March, vol. 3 and Carazo, Salas et al. Nature Cell Biology 2001, March, vol.3) of microtubules is damaged and the cell cannot divide itself. As soon as this cell cannot divide, it dyes. So, the growth of a tumor is inhibited. These fusion proteins can also additionally contain GFP or fluorescent region for optic observation of their action or in order to measure the concentration according to the intensity of the fluorescence. They can also contain hinge regions such as preferably five glyome region or at least one GST tag, His tag or any other tag for affinity purification.

### BRIEF DESCRIPTION OF THE SEQUENCES

In the Seq. 1a is shown the amino acids sequence of the fusion protein SPA-5G-gephyrin.

In the Seq. 1b is shown the nucleic acid sequence encoding this fusion protein.

In the Seq. 2a is shown the amino acid sequence of the fusion protein SPA-5G -Microtubule-binding protein (MBP).

In the Seq. 2b is shown the nucleic acid sequence encoding the fusion protein in the Seq. 2a.

In the Seq. 3 is shown the nucleic acid sequence encoding the fusion protein SPA -5G-FLJ 31424 fis.

In the Seq. 4 is shown the nucleic acid sequence encoding the fusion protein IL 15-5G-Gephyrin-Fc.

In the Seq. 5 is shown the nucleic acid sequence encoding the fusion protein IL 2-5G-Gephyrin-Fc.

In the Seq. 6 is shown the nucleic acid sequence encoding the fusion protein IL 15-5G-MBP-Fc.

In the Seq. 7 is shown the nucleic acid sequence encoding the fusion protein IL 2-5G-MBP-Fc.

In the Seq. 8 is shown the nucleic acid sequence encoding the fusion protein IL 15-5G-FLJ 31424 fis-Fc.

In the Seq. 9 is shown the nucleic acid sequence encoding the fusion protein IL 2-5G-FLJ 314424 fis-Fc.

In the Seq. 10 is shown the nucleic acid sequence encoding the fusion protein SPA-5G -MBP-Fc.

In the Seq. 11 is shown the nucleic acid sequence encoding the fusion protein SPA-5G -Gephyrin-Fc.

In the Seq. 12 is shown the nucleic acid sequence encoding the fusion protein SPA-5G -FLJ 314424 fis-Fc.

The action of the fusion proteins in the sequences la-3 is based on their ability to bind and thereby modif each random therapeutically active antibody.

After binding of fusion proteins to antibodies, fusion protein-antibody complexes arise and they can bind according antigens and after following penetration and internalisation, they bind and grip microtubules and thereso they inhibit-cell division of malignant cells.

The mechanism of action of fusion proteins in the sequences 4-9 is based on the selective targeted binding their antigens or cell or tumour-specific receptors, and after this follows unfolding the double action of microtubule binding regions and additionally the trigger of immune reactions of macrophages.

The mechanism of action of fusion proteins in the sequences 10-12 is based on the modification of antibodies: the complexes possess double action i.e. the ability to bind microtubules and additionally to induce the immune reaction via activation of macrophages.

The DNA encoding for Fc region of the human immunoglobulin G is described by Nakamura, S., Sakugi, I., Kitai, K and Ichikawa, Y (NCBI) http:/www.ncbi.nlm. mnh.gov/entrez . . . NCBI) Sequence Viewer).

The human MRNA for PMBP (putative microtubuli-binding protein) is described by Nadezhdina E. S., and also to find in the NCBI Sequence Viewer.

The mRNA encoding for Homo sapiens Gephyrin (GPH) is described in NCBI.

The mRNA for Homo sapiens Interleukin-2 is described by Chikara, S. K. and Sharma G and is to find in the NCBI Sequence Viewer.

The Homo Sapiens MRNA for Interleukin 15 is described by Sorel, M. A. and Jacques, Y, is expressed in human keratinacytes and is to find in the NCBI Sequence Viewer.

The Homo sapiens cDNA for FLJ 31421 fis encodes a microtubule binding protein, is described by Ota et al and Tashiro et al and is to find in NCBI Sequence Viewer.

The gene sequence encoding for SPA, the DNA encoding for Staphylococcal protein A is described by El-Sayed, A., Alber, J., Laemmer et al and is also described in NCBI Sequence Viewer.

The nucleic acids can be obtained from the authors. Alternatively, the sequences can be isolated according to published methods and protocols using PCR and RT (Reverse Transcriptase) PCR with according primers, and than they will be amplified and cloned.

The methods are known by the specialists.

The cloning and expression of constructs will be preferably carry out in E. coli. The fresh E. coli cell culture will be prepared by the add of 75 mM CaCl₂ (steril, cold 250 ml) and Glycerin (steril, cold 5 mM).

By the carry out, the medium will be firstly heated to 37° C., treated with 8 ml of fresh E. coli culture and by 37° C. strongly shaked and vibrated. After this, the culture will be cooled. The cells will be centrifigated 10 min. by 6000 rpm in GSA-Rotor of a sorvall-centrifuge and suspended in 200 ml of an ice-cold CaCl₂ (875 mM). Than, the cells will be set again on ice for appox. 20 min and again centrifuged 10 min. by 6000 rpm in GSA-Rotor/Sorvall-centrifuge).

The cells will be resuspend in 20 ml ice-cold CaCl₂ (75 mM) and again set on ice for approx. 15 min. 4.2 ml Glycerin will added and mixed. The solution will be filled into steril Eppendof receptacles to 0.5 ml and frozen by −70° C.

The transformation of E-coli will be carry out with 20 microliters ligation solution (or max. 0.5 microliters of a plasmid preparation, that will be 10 times diluted/thinned) 100 ml of competent E-coli cells will be added.

Than, these cells will be kept approx. 30 min on ice (approx. 1 hour), than 2-3 min. heated to 42-43° C. for triggering heat shock and then again set on ice. The cells will be plated for example on X-Gal.

The PCR occurs with the following PCR-Mix for each reagence unit: 9 microliters H 20, 10 ml 10×PCR buffer, 10 microliters dNTP-Mix (2-2.5 mM each), 5 ml BSA (20 mg/ml) or 5 microliter H₂O additionally, 2 ml 5′-Primer (20 pmol/microliter); 2 ml 3,-primer (20 pmol/microliter) and 2 ml Taq-polymerase (54/microliter); at all 40 microliters.

A small quantity of cells will be removed from the plate and distributed or spread on the inner wall in a 1.5 ml Eppendorf receptacle.

After this, the probes will be set into 600 W microwave for 2 min. with open Eppendor. receptacles.

Than, 200 microliters water will be added and good vortexed for resuspending. Than they will be centrifuged 1 min in an Eppendorf centrifuge. From the overlayer will be removed 60 microliters and put into an Eppendorf receptacle as well as 40 microliter PCR mix will be added.

The PCR will be started. The cycles are usual and are dependent on the temperature for a primer and length of the DNA-fragment, for example

| | | | | | | |
|---|---|---|---|---|---|---|
| 94° C. | 2 min | 45° C. | 1 min | 72° C. | 2 min | 1x |
| 94° C. | 30 sec | 45° C. | 30 sec | 72° C. | 2 min | 4x |
| 94° C. | 30 sec | 58° C. | 30 sec | 72° C. | 2 min | 32x |
| 94° C. | 30 sec | 58° C. | 30 sec | 72° C. | 5 min | 1x |

The ligation will be made with 20 microliter starts: 15 microliters of the ligating DNA in water, 4 microliter 5×ligase buffer with PEG 1 microliter T4 DNA-ligase (IU/microliter), in all 20 microliters sticky end-ligations 1-2.5 h by room temperature and blunt-end ligations 4 hours by room temperature. Ligase buffer is consisting from 200 mM tris/HCI pH 7.6, 50 mM Mg Cl 2, 25% PEG 6000(Sigmna or Serva) or PEG 8000, 5 mM ATP and 5 mM DTT.

The E. coli will so be transformed.

### EXAMPLES

### Example 1

Cloning and Expression of the Fusion Construct N-IL-15-L-Gephyrin-Fe-C cDNA for gephyrin will be cloned with PCR or human gephyrin (GPH) mRNA will be cloned through RT-PCR. The data for GPH mRNA sequence are available by the National Center for Biotechnology Information, NIH, Bethesda, Md., 20 894, USA as well as they are to find on the internet page of NCBI (http://www.ncbi.Nlm.nih.gov).

The IL-15 mRNA as well as Fc of IgG mRNA will be also cloned using RT-PCR.

The fusion protein will be constructed from PCR products. The PCR primers are so constructed, that they possess restriction sites on 5′ and 3′ ends for further carry out of ligation steps. The 5′ and 3′ ends of the IL-15 PCR product contain Bam HI and Hind III restriction sites. The 5′ and 3′ ends of the Gephyrin-PCR-product contain Eco RI and Kpn I restriction sites and the 5′ and 3′ ends of the Fc-PCR product contain Pst I and Sac I restriction sites.

The ligation of the IL-15, Gephyrin and Fe sequences in the pUC 19 (2686 bp) vector occurs under standard conditions. The puc 19 vector will be firstly treated with Bam II and Hind 3.

The IL-15 segment will be inserted into the vector through this treatment, pUC 19-IL-15 will be treated with EcoRI and Kpn I for inserting the gephyrin segment. The pUC 19-IL -15-gephyrin vector will than treated with Pst I and Sac I for inserting of the Fe segment into the vector.

The ligation buffer will be mixed and contain 66 mM this, pH 7, 6, 5 mm, 5 mm DTT and 1 mM ATP as well as 20 microliters T4 DNA ligase.

The ligation product will be transformed into E. coli, expressed and than purified.

### Example 2

Cloning and Expression of the Fusion Construct SPA-5G-Gephyrin cDNA for gephyrin will be cloned using PCR, the human gephyrin (GPH) mRNA will be cloned using RT-PCR. The data for the mRNA GPH sequence can be obtained in the internet, by the National Center for Biotechnology Information, NIH, Bethesda, Md., 20 894, (http://www.ncbi.nlm.nihgov:zo/entez/. . . eotide . . . ).

cDNA for SPA ligated with the primer encoding the Five-Glycine-Space will be also cloned using PCR.

The fusion protein will be constructed from the PCR products. The PCR-primes are constructed, that they contain restriction sites on 5′ and 3′ ends for further carry out of ligation steps.

The 5′ and 3′ ends on the gephyrin PCR-product contain Barn HI and Hind III restriction sites. The 5′ and 3′ ends of the SPA-PCR product contain XmnI and Bg III restriction sites. After amplification and purification, the PCR products will be ligated or inserted into PCR H vectors.

Positive clones will be identified through screening of plasmids of the according mass. The clones will be proved and confirmed via DNA sequencing and standard methods.

The gephyrin PCR product will be cleaved after the PCR H through restrictive cleavage using Bam HI and Hind III from the vector and the SPA-PCR product will be cleaved out from the PCR II using Xmn I and Bg III.

The ligation of the gephyrin and SPA segments in the P Mal-C2 expression vector occurs under standard conditions. The p Mal-C2 vector will be treated with Bam HI and Hind 3.

The gephyrin segment will be inserted into the p Mal-C2 vector after this treatment. P Mal-C2-Gephyrin will be cleaved using restriction endonucleases X mn I and Bam HI for inserting the SPA segment.

The ligation buffer will be mixed and contain 66 mM Tris pH 7,6,5 mM Mg Cl, 2.5 mM DTT and 1 mM ATP, as well as T4 DNA ligase (in all 20 microliters).

The ligation will be carry out by 14° C.

The ligation product will be transformed into E. coli, expressed and than purified. With methods described in the Examples 1 and 2 as well as with other methods known by specialists, sequences 1-12 will be cloned and expressed.

1-10. (canceled)

11. Fusion proteins comprising:
regions selected from the group consisting of specific antigen binding regions, microtubule binding regions, and immune response triggering regions.

12. The fusion proteins of claim 11 wherein the specific antigen binding regions comprise a ligand or a ligand region.

13. The fusions proteins of claim 11 wherein the specific antigen binding regions include a component selected from the group consisting of EGF, FGF, CSF, MGF, IL-15, IL-2, and regions thereof.

14. The fusions proteins of claim 11 wherein the specific antigen binding regions include a component selected from the group consisting of gephyrin, Tau, MAP, MID-1, MBP, put MBD or PMBP, FLJ 31424 Fis and regions thereof.

15. The fusions proteins of claim 11 wherein the immune response triggering regions include a component selected from the group consisting of Fc of IgG, B 7.1, B 7.2 and regions thereof.

16. The fusion proteins of claim 11 further comprising spacer or linker regions.

17. The fusion proteins of claim 16 wherein the spacer regions include polyglycine, polyproline, prolines, or glycines.

18. The fusion proteins of claim 11 further comprising at least one component selected from the group consisting of nucleic acid binding regions; polysaccharide binding regions; GFP, fluorescent regions; membrane penetration domains; GST regions; His tags, and regions for carry out of affinity purification a cleavage site for a protease.

19. The fusion proteins of claim 18 wherein the at least one polysaccharide binding region comprises a cellulose-binding region of the CipA protein.

20. The fusion proteins of claim 18 wherein the GFP, fluorescent regions or the membrane penetration domains include the Gene-3-Protein of the bacteriophage fd, gp 41 or Tat protein of the HIV-1.

21. Nucleic Acid and Amino Acid sequences, DNA vectors, amino and nucleic acid, and cloning and expression systems for the fusion proteins of claim 11.

22. Fusion proteins comprising:
regions selected from the group consisting of antibody binding regions, and microtubule-binding regions.

23. The fusion proteins of claim 22 wherein the antibody binding regions include a component selected from the group consisting of Staphylococcal protein A (SPA), extracellular region of the Fc receptor CD 64, and regions thereof.

24. The fusion proteins of claim 22 wherein the microtubule-binding regions include a component selected from the group consisting of Tau, MAP, MID-1, MBP, FL J 31424 Fis, and regions thereof.

25. Fusion proteins according to claim 22 further comprising immune triggering regions.

26. The fusion proteins of claim 25 wherein the immune triggering regions include Fc regions of immunoglobulin G or IgG, HLA-B 7.1 or HLA-B 7.2.

27. The fusion proteins of claim 22 further comprising at least one component selected from the group consisting of spacer or linker regions; nucleic acid binding regions; polysaccharide binding regions; GFP, fluorescent regions; membrane penetration domains; GST regions; His tags, and regions for carry out of affinity purification.a cleavage site for a protease.

28. The fusion proteins of claim 27 wherein:
the polysaccharide binding region comprises a cellulose-binding region of the CipA protein;
the GFP, fluorescent regions or the membrane penetration domains include the Gene-3-Protein of the bacteriophage fd, gp 41 or Tat protein of the HIV-1; and
the spacer regions include polyglycine, polyproline, prolines, or glycines.

29. Nucleic Acid and Amino Acid sequences, DNA vectors, amino and nucleic acid, and cloning and expression systems for the fusion proteins of claim 22.

30. A nucleic acid or amino acid sequence comprises a sequence selected from the group of sequences consisting of SEQ ID NO: 1-12.


SEQUENCE LISTING (14 sequences)

SEQ ID NO: 1 - protein, 676 aa, Artificial
  1a SPA-5G-gephyrin
  FUSION_PRT (1)..(676): fusion protein Staph. aureus Protein A and H. sapiens gephyrin
AAQHDEAQQNAFYQVLNMPNLNADQRNGFIQSLKDDPSQSANVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNGGGGGMSPFPLTSMDKAFITVLEMTPVLGTEIINYRDGMGRVLAQDVYAKDNLPPFPASVKDGYAVRAADGPGDRFIIGESQAGEQPTQTVMPGQVMRVTTGAPIPCGADAVVQVEDTELIRESDDGTEELEVRILVQARPGQDIRPIGHDIKRGECVLAKGTHMGPSEIGLLATVGVTEVEVNKFPVVAVMSTGNELLNPEDDLLPGKIRDSNRSTLLATIQEHGYPTINLGIVGDNPDDLLNALNEGISRADVIITSGGVSMGEKDYLKQVLDIDLHAQIHFGRVFMKPGLPTTFATLDIDGVRKIIFALPGNPVSAVVTCNLFVVPALRKMQGILDPRPTIIKARLSCDVKLDPRPEYHRCILTWHHQEPLPWAQSTGNQMSSRLMSMRSANGLLMLPPKTEQYVELHKGEVVDVMVIGRL

SEQ ID NO: 2 - DNA, 2092 bp, Artificial
  1b SPA-5G-gephyrin
  misc_recomb (1)..(2092): nucleic acid encoding Staph. aureus Protein A and H. sapiens gephyrin fusion prt
tgctgcgcaacacgatgaagctcaacaaaacgctttttatcaagtcttaaatatgcctaacttaaatgctgatcaacgcaatggttttatccaaagccttaaagatgatccaagccaaagtgctaacgttttaggtgaagctaaaaaattaaacgaatctcaagcaccgaaagctgacaacaatttcaacaaagaacaacaaaatgctttctatgaaatcttgaacatgcctaacttgaacgaagaacaacgcaatggtttcatccaaagcttaaaagatgacccaagtcaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagagcaacgtaacggcttcatccaaagccttaaagacgatccttcagtgagcaaagaaattttagcagaagctaaaaagctaaacgatgctcaagcaccaaaagaggaagacaacaacaaacctggtaaagaagacggcaacaaacctggcaaagaagacggtaacggcggcggcggcggcgtttaggtcacagtgctgtcgatatcaccaaggtggctagaagacatcgcatgtctccttttcctctgacatctatggacaaagcctttatcacagtcctggagatgactccggtgcttgggacagaaatcatcaattaccgagatggaatggggcgagtccttgctcaagatgtatatgcaaaagacaatttaccccccttcccagcatcagtaaaagatggctatgctgtccgagctgctgatggcccaggagatcgtttcatcattggggaatcccaagctggtgaacagccaactcagacagtaatgccaggacaagtcatgcgggttacaacaggtgctccaataccctgcggtgctgatgcagtagtacaagtggaagataccgaacttatcagggaatcagatgatggcactgaagaacttgaagtgcgaattctggtgcaagctcggccaggccaagatatcagacccatcggccatgacattaaaagaggggaatgtgttttggccaaaggaacccacatgggcccctcagagattggtcttctggcaactgtaggtgtcacagaggttgaagttaataagtttccagtggttgcagtcatgtcaacagggaatgagctgctaaatcctgaagatgacctcttaccagggaagattcgagacagcaatcgttcaactcttctagcaacaattcaggaacatggttaccccacgatcaacttgggtattgtaggagacaacccagatgacttactcaatgccttgaatgagggtatcagtcgtgctgatgtcatcatcacatcagggggtgtatccatgggggaaaaggactatctcaagcaggtgctggacattgatcttcatgctcagatccattttggcagggtttttatgaaaccaggcttgccaacaacatttgcaactttggatattgatggtgtaagaaaaataatctttgcactacctgggaatcctgtatcggctgtggtcacctgcaatctctttgttgtgcctgcactgaggaaaatgcagggcatcttggatcctcggccaaccatcatcaaagcaaggttatcatgtgatgtaaaacttgatcctcgtccagaataccatcggtgtatactaacttggcatcaccaagaaccactaccttgggcacagagtacaggtaatcaaatgagcagccgtctgatgagcatgcgcagtgccaatggattgttgatgctacctccaaagacagaacagtacgtggagctccacaaaggcgaggtggtggatgtcatggtcattggacggctatgatggtcaccag

SEQ ID NO: 3 - protein, 300 aa, Artificial
  2a SPA-5G-MBP
  FUSION_PRT (1)..(300): Fusion Protein of Staph. aureus Protein A and H. sapiens MBP
  MISC_FEATURE (264)..(264): Xaa can be any naturally occurring amino acid
  MISC_FEATURE (278)..(278): Xaa can be any naturally occurring amino acid
  MISC_FEATURE (281)..(281): Xaa can be any naturally occurring amino acid
AAQHDEAQQNAFYQVLNMPNLNADQRNGFIQSLKDDPSQSANVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNGGGGGAAASTAXASTAKETAEAVADXILXKAGPLVAVSAVALDITAYP

SEQ ID NO: 4 - DNA, 912 bp, Artificial
  2b SPA-5g-MBP
  misc_recomb (1)..(912): nucleic acid encoding Staph. aureus Protein A and H. sapiens MBP fusion prt
  misc_feature (792)..(792): n is a, c, g, t or u
  misc_feature (835)..(835): n is a, c, g, t or u
  misc_feature (844)..(844): n is a, c, g, t or u
tgctgcgcaacacgatgaagctcaacaaaacgctttttatcaagtcttaaatatgcctaacttaaatgctgatcaacgcaatggttttatccaaagccttaaagatgatccaagccaaagtgctaacgttttaggtgaagctaaaaaattaaacgaatctcaagcaccgaaagctgacaacaatttcaacaaagaacaacaaaatgctttctatgaaatcttgaacatgcctaacttgaacgaagaacaacgcaatggtttcatccaaagcttaaaagatgacccaagtcaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagagcaacgtaacggcttcatccaaagccttaaagacgatccttcagtgagcaaagaaattttagcagaagctaaaaagctaaacgatgctcaagcaccaaaagaggaagacaacaacaaacctggtaaagaagacggcaacaaacctggcaaagaagacggtaacggcggcggcggcggcgcggccgcgtcgaccgcggncgcgtcgacggcaaaggagactgctgaggctgttgctgatganatactgganaaggctgggccacttgttgctgtgtctgctgttgcacttgatataactgcctacccctaaaagccaaa

SEQ ID NO: 5 - DNA, 3718 bp, Artificial
  3 SPA-5g-FLJ 314424 fis (MBP)
  misc_recomb (1)..(3718): nucleic acid encoding Staph. aureus Protein A and H. Sapiens FLJ 314424 fis fusion prt
tgctgcgcaacacgatgaagctcaacaaaacgctttttatcaagtcttaaatatgcctaacttaaatgctgatcaacgcaatggttttatccaaagccttaaagatgatccaagccaaagtgctaacgttttaggtgaagctaaaaaattaaacgaatctcaagcaccgaaagctgacaacaatttcaacaaagaacaacaaaatgctttctatgaaatcttgaacatgcctaacttgaacgaagaacaacgcaatggtttcatccaaagcttaaaagatgacccaagtcaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagagcaacgtaacggcttcatccaaagccttaaagacgatccttcagtgagcaaagaaattttagcagaagctaaaaagctaaacgatgctcaagcaccaaaagaggaagacaacaacaaacctggtaaagaagacggcaacaaacctggcaaagaagacggtaacggcggcggcggcggcaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgatt

SEQ ID NO: 6 - DNA, 2553 bp, Artificial
  4 IL 15-5G-gephyrin-Fc
  misc_recomb (1)..(2553): nucleic acid encoding H. sapiens IL 15 and H. sapiens gephyrin fusion protein
ccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactagaggaaaaaaatattaaagaatttttgcagagttttgtacatattgtccaaatgttcatcaacacttcttggcggcggcggcggcgtttaggtcacagtgctgtcgatatcaccaaggtggctagaagacatcgcatgtctccttttcctctgacatctatggacaaagcctttatcacagtcctggagatgactccggtgcttgggacagaaatcatcaattaccgagatggaatggggcgagtccttgctcaagatgtatatgcaaaagacaatttaccccccttcccagcatcagtaaaagatggctatgctgtccgagctgctgatggcccaggagatcgtttcatcattggggaatcccaagctggtgaacagccaactcagacagtaatgccaggacaagtcatgcgggttacaacaggtgctccaataccctgcggtgctgatgcagtagtacaagtggaagataccgaacttatcagggaatcagatgatggcactgaagaacttgaagtgcgaattctggtgcaagctcggccaggccaagatatcagacccatcggccatgacattaaaagaggggaatgtgttttggccaaaggaacccacatgggcccctcagagattggtcttctggcaactgtaggtgtcacagaggttgaagttaataagtttccagtggttgcagtcatgtcaacagggaatgagctgctaaatcctgaagatgacctcttaccagggaagattcgagacagcaatcgttcaactcttctagcaacaattcaggaacatggttaccccacgatcaacttgggtattgtaggagacaacccagatgacttactcaatgccttgaatgagggtatcagtcgtgctgatgtcatcatcacatcagggggtgtatccatgggggaaaaggactatctcaagcaggtgctggacattgatcttcatgctcagatccattttggcagggtttttatgaaaccaggcttgccaacaacatttgcaactttggatattgatggtgtaagaaaaataatctttgcactacctgggaatcctgtatcggctgtggtcacctgcaatctctttgttgtgcctgcactgaggaaaatgcagggcatcttggatcctcggccaaccatcatcaaagcaaggttatcatgtgatgtaaaacttgatcctcgtccagaataccatcggtgtatactaacttggcatcaccaagaaccactaccttgggcacagagtacaggtaatcaaatgagcagccgtctgatgagcatgcgcagtgccaatggattgttgatgctacctccaaagacagaacagtacgtggagctccacaaaggcgaggtggtggatgtcatggtcattggacggctatgatggtcaccagctgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 7 - DNA, 2505 bp, Artificial
  5 IL 2-5G-gephyrin -Fc
  misc_recomb (1)..(2505): nucleic acid encoding H. sapiens IL 2, H. sapiens gephyrin and H. sapiens IgG Fc fusion protein
atgcctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactgacttgataaggcggcggcggcggcgtttaggtcacagtgctgtcgatatcaccaaggtggctagaagacatcgcatgtctccttttcctctgacatctatggacaaagcctttatcacagtcctggagatgactccggtgcttgggacagaaatcatcaattaccgagatggaatggggcgagtccttgctcaagatgtatatgcaaaagacaatttaccccccttcccagcatcagtaaaagatggctatgctgtccgagctgctgatggcccaggagatcgtttcatcattggggaatcccaagctggtgaacagccaactcagacagtaatgccaggacaagtcatgcgggttacaacaggtgctccaataccctgcggtgctgatgcagtagtacaagtggaagataccgaacttatcagggaatcagatgatggcactgaagaacttgaagtgcgaattctggtgcaagctcggccaggccaagatatcagacccatcggccatgacattaaaagaggggaatgtgttttggccaaaggaacccacatgggcccctcagagattggtcttctggcaactgtaggtgtcacagaggttgaagttaataagtttccagtggttgcagtcatgtcaacagggaatgagctgctaaatcctgaagatgacctcttaccagggaagattcgagacagcaatcgttcaactcttctagcaacaattcaggaacatggttaccccacgatcaacttgggtattgtaggagacaacccagatgacttactcaatgccttgaatgagggtatcagtcgtgctgatgtcatcatcacatcagggggtgtatccatgggggaaaaggactatctcaagcaggtgctggacattgatcttcatgctcagatccattttggcagggtttttatgaaaccaggcttgccaacaacatttgcaactttggatattgatggtgtaagaaaaataatctttgcactacctgggaatcctgtatcggctgtggtcacctgcaatctctttgttgtgcctgcactgaggaaaatgcagggcatcttggatcctcggccaaccatcatcaaagcaaggttatcatgtgatgtaaaacttgatcctcgtccagaataccatcggtgtatactaacttggcatcaccaagaaccactaccttgggcacagagtacaggtaatcaaatgagcagccgtctgatgagcatgcgcagtgccaatggattgttgatgctacctccaaagacagaacagtacgtggagctccacaaaggcgaggtggtggatgtcatggtcattggacggctatgatggtcaccagctgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 8 - DNA, 1373 bp, Artificial
  6 IL 15-5G-MBP-Fc
  misc_recomb (1)..(1373): nucleic acid encoding H.sapiens IL 15, H.sapiens MBP and H.sapiens IgG Fc fusion prt
  misc_feature (488)..(488): n is a, c, g, t or u
  misc_feature (531)..(531): n is a, c, g, t or u
  misc_feature (540)..(540): n is a, c, g, t or u
ccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactagaggaaaaaaatattaaagaatttttgcagagttttgtacatattgtccaaatgttcatcaacacttcttggcggcggcggcggcgcggccgcgtcgaccgcggncgcgtcgacggcaaaggagactgctgaggctgttgctgatganatactgganaaggctgggccacttgttgctgtgtctgctgttgcacttgatataactgcctacccctaaaagccaaactgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 9 - DNA, 1325 bp, Artificial
  7 IL 2-5G-MBP-Fc
  misc_recomb (1)..(1325): nucleic acid encoding H.sapiens IL 2, H.sapiens MBP and H.sapiens IgG Fc fusion prt
  misc_feature (440)..(440): n is a, c, g, t or u
  misc_feature (483)..(483): n is a, c, g, t or u
  misc_feature (492)..(492): n is a, c, g, t or u
atgcctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactgacttgataaggcggcggcggcggcgcggccgcgtcgaccgcggncgcgtcgacggcaaaggagactgctgaggctgttgctgatganatactgganaaggctgggccacttgttgctgtgtctgctgttgcacttgatataactgcctacccctaaaagccaaactgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 10 - DNA, 4178 bp, Artificial
  8 IL 15-5G-FLJ 314424 fis (MBP)-Fc
ccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactagaggaaaaaaatattaaagaatttttgcagagttttgtacatattgtccaaatgttcatcaacacttcttggcggcggcggcggcaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattctgttgacaattaatcatcggctcgtataatgtgtggaattgtgagcggataacaatttcacacaggaaacaggatccgataatgacatgcccaccgtgcccagcacctgaactcctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 11 - DNA, 4131 bp, Artificial
  9 IL 2-5G-FLJ 314424 fis-Fc
atgcctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactgacttgataaggcggcggcggcggcaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattctgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 12 - DNA, 1677 bp, Artificial
  10 SPA-5G-MBP-Fc
  misc_feature (792)..(792): n is a, c, g, t or u
  misc_feature (835)..(835): n is a, c, g, t or u
  misc_feature (844)..(844): n is a, c, g, t or u
tgctgcgcaacacgatgaagctcaacaaaacgctttttatcaagtcttaaatatgcctaacttaaatgctgatcaacgcaatggttttatccaaagccttaaagatgatccaagccaaagtgctaacgttttaggtgaagctaaaaaattaaacgaatctcaagcaccgaaagctgacaacaatttcaacaaagaacaacaaaatgctttctatgaaatcttgaacatgcctaacttgaacgaagaacaacgcaatggtttcatccaaagcttaaaagatgacccaagtcaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagagcaacgtaacggcttcatccaaagccttaaagacgatccttcagtgagcaaagaaattttagcagaagctaaaaagctaaacgatgctcaagcaccaaaagaggaagacaacaacaaacctggtaaagaagacggcaacaaacctggcaaagaagacggtaacggcggcggcggcggcgcggccgcgtcgaccgcggncgcgtcgacggcaaaggagactgctgaggctgttgctgatganatactgganaaggctgggccacttgttgctgtgtctgctgttgcacttgatataactgcctacccctaaaagccaaactgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 13 - DNA, 2857 bp, Artificial
  11 SPA-5G-gephyrin-Fc
  misc_recomb (1)..(2857): nucleic acid encoding Staph aureus Protein A, H.sapiens gephyrin and H.sapiens IgG Fc fusion prt
tgctgcgcaacacgatgaagctcaacaaaacgctttttatcaagtcttaaatatgcctaacttaaatgctgatcaacgcaatggttttatccaaagccttaaagatgatccaagccaaagtgctaacgttttaggtgaagctaaaaaattaaacgaatctcaagcaccgaaagctgacaacaatttcaacaaagaacaacaaaatgctttctatgaaatcttgaacatgcctaacttgaacgaagaacaacgcaatggtttcatccaaagcttaaaagatgacccaagtcaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagagcaacgtaacggcttcatccaaagccttaaagacgatccttcagtgagcaaagaaattttagcagaagctaaaaagctaaacgatgctcaagcaccaaaagaggaagacaacaacaaacctggtaaagaagacggcaacaaacctggcaaagaagacggtaacggcggcggcggcggcgtttaggtcacagtgctgtcgatatcaccaaggtggctagaagacatcgcatgtctccttttcctctgacatctatggacaaagcctttatcacagtcctggagatgactccggtgcttgggacagaaatcatcaattaccgagatggaatggggcgagtccttgctcaagatgtatatgcaaaagacaatttaccccccttcccagcatcagtaaaagatggctatgctgtccgagctgctgatggcccaggagatcgtttcatcattggggaatcccaagctggtgaacagccaactcagacagtaatgccaggacaagtcatgcgggttacaacaggtgctccaataccctgcggtgctgatgcagtagtacaagtggaagataccgaacttatcagggaatcagatgatggcactgaagaacttgaagtgcgaattctggtgcaagctcggccaggccaagatatcagacccatcggccatgacattaaaagaggggaatgtgttttggccaaaggaacccacatgggcccctcagagattggtcttctggcaactgtaggtgtcacagaggttgaagttaataagtttccagtggttgcagtcatgtcaacagggaatgagctgctaaatcctgaagatgacctcttaccagggaagattcgagacagcaatcgttcaactcttctagcaacaattcaggaacatggttaccccacgatcaacttgggtattgtaggagacaacccagatgacttactcaatgccttgaatgagggtatcagtcgtgctgatgtcatcatcacatcagggggtgtatccatgggggaaaaggactatctcaagcaggtgctggacattgatcttcatgctcagatccattttggcagggtttttatgaaaccaggcttgccaacaacatttgcaactttggatattgatggtgtaagaaaaataatctttgcactacctgggaatcctgtatcggctgtggtcacctgcaatctctttgttgtgcctgcactgaggaaaatgcagggcatcttggatcctcggccaaccatcatcaaagcaaggttatcatgtgatgtaaaacttgatcctcgtccagaataccatcggtgtatactaacttggcatcaccaagaaccactaccttgggcacagagtacaggtaatcaaatgagcagccgtctgatgagcatgcgcagtgccaatggattgttgatgctacctccaaagacagaacagtacgtggagctccacaaaggcgaggtggtggatgtcatggtcattggacggctatgatggtcaccagctgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc

SEQ ID NO: 14 - DNA, 4483 bp, Artificial
  12 SPA-5G-FLJ 314424 fis-Fc
  misc_recomb (1)..(4483): nucleic acid encoding Staph. aureus Protein A, H.sapiens FLJ 314424 fis and H.sapiens IgG Fc fusion prt
tgctgcgcaacacgatgaagctcaacaaaacgctttttatcaagtcttaaatatgcctaacttaaatgctgatcaacgcaatggttttatccaaagccttaaagatgatccaagccaaagtgctaacgttttaggtgaagctaaaaaattaaacgaatctcaagcaccgaaagctgacaacaatttcaacaaagaacaacaaaatgctttctatgaaatcttgaacatgcctaacttgaacgaagaacaacgcaatggtttcatccaaagcttaaaagatgacccaagtcaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagagcaacgtaacggcttcatccaaagccttaaagacgatccttcagtgagcaaagaaattttagcagaagctaaaaagctaaacgatgctcaagcaccaaaagaggaagacaacaacaaacctggtaaagaagacggcaacaaacctggcaaagaagacggtaacggcggcggcggcggcaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattctgttgacaattaatcatcggctcgtataatgtgtggaattgtgagcggataacaatttcacacaggaaacaggatccgataatgacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaataataggatcc